SEQUENCE LISTING (18 sequences)

SEQ ID NO: 1 - DNA, 22 bp, synthetic construct
  source 1..22: mol_type = other DNA
agaaggtaagaggctcaccgtg

SEQ ID NO: 2 - DNA, 19 bp, synthetic construct
  source 1..19: mol_type = other DNA
cctcccccgacgaatggac

SEQ ID NO: 3 - DNA, 25 bp, synthetic construct
  source 1..25: mol_type = other DNA
tcgttatgcctttgccactgcccat

SEQ ID NO: 4 - DNA, 18 bp, synthetic construct
  source 1..18: mol_type = other DNA
ggtaagaggctcaccgtg

SEQ ID NO: 5 - DNA, 18 bp, synthetic construct
  source 1..18: mol_type = other DNA
ggtaagcagccccctccg

SEQ ID NO: 6 - DNA, 26 bp, synthetic construct
  source 1..26: mol_type = other DNA
gtcgttatgcctttgccactgcccat

SEQ ID NO: 7 - DNA, 19 bp, synthetic construct
  source 1..19: mol_type = other DNA
tctgccgcctatggatccc

SEQ ID NO: 8 - DNA, 22 bp, synthetic construct
  source 1..22: mol_type = other DNA
cttctcgggttttctttcggaa

SEQ ID NO: 9 - DNA, 24 bp, synthetic construct
  source 1..24: mol_type = other DNA
ctcactcggccaggggttgctttt

SEQ ID NO: 10 - DNA, 18 bp, synthetic construct
  source 1..18: mol_type = other DNA
ccgaaatggcggctcccc

SEQ ID NO: 11 - DNA, 19 bp, synthetic construct
  source 1..19: mol_type = other DNA
gggctggtgattcaaggag

SEQ ID NO: 12 - DNA, 25 bp, synthetic construct
  source 1..25: mol_type = other DNA
cggccaggggttgcttttaaatggg

SEQ ID NO: 13 - DNA, 40 bp, synthetic construct
  source 1..40: mol_type = other DNA
agggaattccccttaccatcttgacaatgtccatagaaga

SEQ ID NO: 14 - DNA, 20 bp, synthetic construct
  source 1..20: mol_type = other DNA
gccttaaaggggttcggacc

SEQ ID NO: 15 - DNA, 40 bp, synthetic construct
  source 1..40: mol_type = other DNA
ttcgaacaatcaggaaacccttgatcactggcttgcggaa

SEQ ID NO: 16 - DNA, 20 bp, synthetic construct
  source 1..20: mol_type = other DNA
acagggccagagggaattcc

SEQ ID NO: 17 - DNA, 20 bp, synthetic construct
  source 1..20: mol_type = other DNA
gccttaaaggggttcggacc

SEQ ID NO: 18 - DNA, 40 bp, synthetic construct; identical to SEQ ID NO: 15
  source 1..40: mol_type = other DNA

(19) United States
(12) Patent Application Publication
(10) Pub. No.: US 2026/0159906 A1
(43) Pub. Date: Jun. 11, 2026

(54) RETROVIRUS-LIKE PARTICLES (RVLP) DETECTION AND QUANTITATION

(71) Applicant: Merck Sharp & Dohme LLC, Rahway, NJ (US)

(72) Inventor: Rosalind Ang, Wayne, NJ (US)

(73) Assignee: Merck Sharp & Dohme LLC, Rahway, NJ (US)

(21) Appl. No.: 19/408,934

(22) Filed: Dec. 4, 2025

Related U.S. Application Data
(60) Provisional application No. 63/730,564, filed on Dec. 11, 2024.

(51) Int. Cl. C12Q 1/70; C12N 15/10; C12Q 1/6851; C12Q 1/686

(52) U.S. Cl. CPC C12Q 1/701; C12N 15/1096; C12Q 1/6851; C12Q 1/686

(57) ABSTRACT

A real time quantitative reverse transcriptase polymerase chain reaction (RT-qPCR) for detecting and quantitating RVLP particles is provided.

### CROSS-REFERENCE TO RELATED APPLICATIONS

This application claims the benefit of U.S. Provisional Patent Application Ser. No. 63/730,564 filed Dec. 11, 2024, the entire contents of which are incorporated by reference herein.

### REFERENCE TO SEQUENCE LISTING SUBMITTED ELECTRONICALLY

The contents of the electronic sequence listing (26115-US-PCT_SL.xml; Size: 16,744 bytes; and Date of Creation: Oct. 23, 2025) are herein incorporated by reference in their entirety.

### FIELD

This disclosure relates generally to detection and quantitation of retrovirus-like particles (RVLP) using reverse transcription-based real-time polymerase chain reaction (RT-qPCR).

### BACKGROUND

Retrovirus-like particles (RVLP) are Type C proviruses that have been found to be produced by Chinese Hamster Ovary (CHO) cells and in Chinese hamster liver. These particles have been observed to be present in products such as therapeutic proteins produced using CHO host cells (Lieber M M, et al., Science 182(4107) 1973: 56-59; Lubiniecki A S, May L H., Dev. Biol. Stand. 60, 1985: 141-146). Though these RVLPs are apparently defective and non-infectious, because of their morphology and biochemical resemblance to tumorigenic retroviruses, RVLP pose a theoretical safety concern. Hence, RVLP viral clearance validation is a regulatory requirement for worldwide product market registration of active pharmaceutical products derived from CHO host cell (ICH_Q5A(R2) Step2_draft_Guideline_2022_0826.pdf).

The standard method for detecting and quantitating RVLP is by using Transmission Electron Microscopy (TEM) in which a beam of electrons is focused on a test specimen to produce a highly magnified and detailed image of the specimen. The identification and quantitation of RVLP particles is performed based on the size and morphological characteristics observed in the image. The method lacks specificity. Additionally, the method requires specialized expertise and instruments, is associated with high cost and long lead times, and is not capable of detecting below 1×10⁶ particles/mL (limit of detection), which is often the case in permeate from continuous manufacturing.

Each RVLP particle contains two copies of the viral genomic particle RNA (pRNA) molecule. An alternative to the TEM method for detecting and quantitating RVLPs is to use nucleic acid-based techniques, specifically, reverse transcription—real time quantitative Polymerase Chain Reaction (RT-qPCR). RT-qPCR requires reverse transcription of genetic material from RVLP, i.e., RNA, into cDNA prior to PCR amplification. The PCR amplification step involves multiple reiterative cycles (generally 40 cycles) and requires the use of a thermostable DNA polymerase and a pair of primers (synthetic oligonucleotides—forward and reverse primers) to direct the synthesis of DNA from deoxynucleotide substrates in the region of interest of the single-stranded DNA template (derived from the cDNA). The outcome of each cycle is a copy of synthesized DNA (i.e., amplicon) complementary to the region of interest on the single-stranded DNA template. The quantity of amplicons can be visualized on a gel or via dye/fluorescence-based approaches that additionally requires a fluorescence probe. The specificity and performance of the PCR method (i.e., sensitivity) is dictated by oligonucleotide sequence of the primer and probe set.

Based on the identified nucleotide sequence of RVLP by Lie et al. (Genebank: U09104.1), an RT-qPCR method, which incorporates the use of a custom designed primer set and TaqMAN® probe to detect and quantitate RVLP particles has been reported (De Wit C, et al., Biologicals 28(3), 2000: 137-148). The TaqMAN® probe contains an oligonucleotide sequence complementary to a segment within the region targeted by the primer set, is tagged with both reporter and quencher (does not fluoresce upon excitation). Upon synthesis of the amplicon, the reporter is released from the probe by DNA polymerase, thereby enabling it to fluoresce upon excitation. The signal intensity of the probe is measured after each PCR cycle and is directly proportional to the accumulative amplicons generated.

Because of the variation of the RVLP genome in different CHO cell lines, the primer-probe set used in De Wit C, et al., 2000, may not be reliable for detecting or quantitating of RVLP in all CHO cell lines. Thus, there is a need to identify primer-probe sets that can be used reliably for detection or quantitation of RVLP in all or the vast majority of CHO cell lines in use.

### SUMMARY

Accordingly, the present disclosure provides a primer-probe set that can be used reliably for detection and quantitation of RVLP in a vast majority of CHO cell lines using RT-qPCR.

In one aspect, the disclosure provides a method for detecting retrovirus-like particles (RVLP) in a host cell culture using reverse transcription based real-time polymerase chain reaction (PCR) (RT qPCR), the method comprising (a) reverse transcribing genomic RNA from the RVLP to cDNA and (b) amplifying and quantifying the cDNA using a pair of primers and a probe, each primer pair and probe having sequences selected from the group consisting of:

(i)
(SEQ ID NO: 4)
5′-GGTAAGAGGCTCACCGTG-3′ (forward primer),
(SEQ ID NO: 5)
5′-GGTAAGCAGCCCCCTCCG-3′ (reverse primer),
(SEQ ID NO: 6)
5′-GTCGTTATGCCTTTGCCACTGCCCAT-3′ (probe);
and
(ii)
(SEQ ID NO: 10)
5′-CCGAAATGGCGGCTCCCC-3′ (forward primer),
(SEQ ID NO: 11)
5′-GGGCTGGTGATTCAAGGAG-3′ (reverse primer),
(SEQ ID NO: 12)
5′-CGGCCAGGGGTTGCTTTTAAATGGG-3′ (probe).

The summary of the technology described above is non-limiting and other features and advantages of the technology will be apparent from the following detailed description, and from the claims.

### BRIEF DESCRIPTION OF THE DRAWINGS

FIG. 1 is a graph showing performance of primer-probe sets 2, 4, and Current, with multiple dilutions of harvested clarified culture fluid (HCCF) used as the sample.

FIG. 2 is a graph showing performance of primer-probe sets 2, 4, and Current, with different levels of CHO genomic DNA used as the sample.

FIG. 3 is a graph showing performance of primer-probe sets 1, 3, 4, and 6, with different levels of CHO genomic DNA being used as the sample. Cycle thresholds for primer-probe sets 1, 3 and 6 that were higher than 37, which was taken to indicate lack of amplification, are not shown in the Figure.

### DETAILED DESCRIPTION

The present disclosure is directed to Detection and quantitation of RVLP particles reverse transcriptase—real time quantitative Polymerase Chain Reaction (RT-qPCR).

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” in quantitative terms refers to plus or minus 10% of the value it modifies (rounded up to the nearest whole number if the value is not sub-dividable, such as a number of molecules or nucleotides).

All ranges disclosed herein are inclusive of the recited endpoint and independently combinable (for example, the range of “from 50 mg to 500 mg” is inclusive of the endpoints, 50 mg and 500 mg, and all the intermediate values). The endpoints of the ranges and any values disclosed herein are not limited to the precise range or value; they are sufficiently imprecise to include values approximating these ranges and/or values.

As used herein, the term “comprising” may include the embodiments “consisting of” and “consisting essentially of” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated components, which allows the presence of only the named components or compounds, along with any acceptable carriers or fluids, and excludes other components or compounds.

### Real Time Quantitative Polymerase Chain Reaction (RT-qPCR)

The RT-qPCR assay used herein is based on the fact that each retrovirus like particle (RVLP) contains two copies of the viral genomic particle RNA (pRNA) molecule. RVLPs may thus be quantified by determining the pRNA copy number. The number of RVLPs in a given sample would be half the pRNA copy number obtained from the assay. The assay uses a nucleic-acid probe complementary to an internal segment of the target DNA that is being amplified. The probe is tagged with two fluorophores. The fluorophores are such that the emission spectrum of one overlaps the excitation spectrum of the other. This results in “quenching” of the first fluorophore by the second. The probe is present during the PCR. If primer extension occurs, i.e., if a product is made, the 5′ to 3′ exonuclease activity of the Taq polymerase causes the probe to be degraded via the 5′-nuclease activity of polymerase. This results in the two fluorophores being separated, quenching is reduced, and the intensity of light emitted is increased. As in De Wit et al. 2000, the primer-probe sets used in this disclosure target the pol region of the CHO type C retrovirus genome (Gen-Bank accession number U09104), but different segments within the region.

The sequences of the various primer-probe sets used are listed below in Table 1.

TABLE 1
Sequences

| | Description: | |
|---|---|---|
| SEQ ID NO: | Primer-Probe Set | Sequence |
| 1 | Set 1 Forward Primer | 5′-AGAAGGTAAGAGGCTCACCGTG-3′ |
| 2 | Set 1 Reverse Primer | 5′-CCTCCCCCGACGAATGGAC-3′ |
| 3 | Set 1 Probe | 5′-TCGTTATGCCTTTGCCACTGCCCAT-3′ |
| 4 | Set 2 Forward Primer | 5′-GGTAAGAGGCTCACCGTG-3′ |
| 5 | Set 2 Reverse Primer | 5′-GGTAAGCAGCCCCCTCCG-3′ |
| 6 | Set 2 Probe | 5′-GTCGTTATGCCTTTGCCACTGCCCAT-3′ |
| 7 | Set 3 Forward Primer | 5′-TCTGCCGCCTATGGATCCC-3′ |
| 8 | Set 3 Reverse Primer | 5′-CTTCTCGGGTTTTCTTTCGGAA-3′ |
| 9 | Set 3 Probe | 5′-CTCACTCGGCCAGGGGTTGCTTTT-3′ |
| 10 | Set 4 Forward Primer | 5′-CCGAAATGGCGGCTCCCC-3′ |
| 11 | Set 4 Reverse Primer | 5′-GGGCTGGTGATTCAAGGAG-3′ |
| 12 | Set 4 Probe | 5′-CGGCCAGGGGTTGCTTTTAAATGGG-3′ |
| 13 | Set 5 Forward Primer | 5′-AGGGAATTCCCCTTACCATCTTGACAATGT |
| | | CCATAGAAGA-3′ |
| 14 | Set 5 Reverse Primer | 5′-GCCTTAAAGGGGTTCGGACC-3′ |
| 15 | Set 5 Probe | 5′-TTCGAACAATCAGGAAACCCTTGATCACTG |
| | | GCTTGCGGAA-3′ |
| 16 | Set 6 Forward Primer | 5′-ACAGGGCCAGAGGGAATTCC-3′ |
| 17 | Set 6 Reverse Primer | 5′-GCCTTAAAGGGGTTCGGACC-3′ |
| 18 | Set 6 Probe | 5′-TTCGAACAATCAGGAAACCCTTGATCACTG |
| | | GCTTGCGGAA-3′ |

### EMBODIMENTS

The invention is further described below in the following embodiments.

In one embodiment, provided herein is a method for detecting or quantifying retrovirus-like particles (RVLP) in a host cell culture using reverse transcription based real-time polymerase chain reaction (PCR) (RT qPCR), the method comprising (a) reverse transcribing genomic RNA from the RVLP to cDNA, and (b) amplifying and quantifying the cDNA using a pair of primers and a probe, each primer pair and probe having sequences selected from the group consisting of:

(i)
(SEQ ID NO: 4)
5′-GGTAAGAGGCTCACCGTG-3′ (forward primer),
(SEQ ID NO: 5)
5′-GGTAAGCAGCCCCCTCCG-3′ (reverse primer),
(SEQ ID NO: 6)
5′-GTCGTTATGCCTTTGCCACTGCCCAT-3′ (probe);
and
(ii)
(SEQ ID NO: 10)
5′-CCGAAATGGCGGCTCCCC-3′ (forward primer),
(SEQ ID NO: 11)
5′-GGGCTGGTGATTCAAGGAG-3′ (reverse primer),
(SEQ ID NO: 12)
5′-CGGCCAGGGGTTGCTTTTAAATGGG-3′ (probe).

In some embodiments, the host cell culture comprises Chinese Hamster Ovary (CHO) cells.

In some embodiments, the CHO cells are deficient in dihydrofolate reductase.

In some embodiments, the primer pair and probe have the sequences shown in paragraph [0023]b(i).

In some embodiments, the primer pair and probe have the sequences shown in paragraph [0023]b(ii).

In some embodiments, the host cell culture is used for expressing a protein. In one embodiment, the protein is pembrolizumab.

### EXAMPLES

The following examples are meant to be illustrative and should not be construed as further limiting. The contents of the figures and all references, patents, and published patent applications cited throughout this application are expressly incorporated herein by reference.

### Example 1: Detection and Quantitation of RVLP Using RT-qPCR

### Methods and Reagents

Reagents for screening of RVLP includes Primer-Probe sets shown in Table 1, CHO genomic DNA stock (Cygnus), TaqMan Fast Virus 1-Step Mastermix (Applied Biosystem), and nuclease free water (Ambion). Altogether, five sets of primer-probe sets (sets 1-5) were custom-designed as shown above in Table 1.

The screening for optimal performance were performed in two stages.

Stage 1: Comparison of primer-probe sets 2 and 4 with the primer-probe set reported by De Wit et al., 2000 (set 6 in Table 1; also denoted as Current). Note that primer-probe sets 2 and 4, each targets a different segment of the RVLP genome, and both target segments are different from that targeted by the primer-probe set, Current. In the early stage of method development, a direct RT-qPCR method described in Hussain et al., 2020 was used to test the performance of the primer-probe sets listed in Table 1.

(A) Screening with CHO DNA stock solution: A 10-fold serial dilution of CHO DNA stock (2−2×10⁶ RVLP copies) was made. Next, the target segment was amplified with TaqMan Fast Virus 1-Step Mastermix containing primer/probe sets of interest. The following thermal cycling condition was used: Stage 1 (reverse transcription): 50° C. for 10 min, 1 cycle; Stage 2 (PCR amplification): 95° C. for 1 minute, 1 cycle; and Stage 3: 40 cycles, each consisting of: step 1: 95° C. for 3 seconds, step 2: 55° C. for 30 seconds, and step 3: 72° C. for 30 seconds.

(B) Screening using harvested clarified culture fluid (HCCF) samples: HCCF is the liquid component obtained after separating cells from the culture medium harvested from a bioreactor in biotechnology processes such as cell culture and fermentation. An aliquot of HCCF, diluted 5-, 50-, and 500-fold, was pretreated with RNase in a qPCR plate at the following thermal cycle conditions: Stage 1: 30° C. for 30 min, 1 cycle; Stage 2: 37° C. for 15 minute, 1 cycle; Stage 3: 95° C. for 10 mins, 1 cycle, and Stage 4: 12° C. hold. Then TaqMan Fast Virus 1-Step Mastermix containing primer-probe set of interest was dispensed into the respective wells and the reverse transcription and PCR amplification of the target segment was carried out using the thermal cycling conditions described above in section A.

Stage 2: Comparing the performance of primer-probe sets 1, 3, 5, and 6 with that of primer-probe set 4.

A 10-fold serial dilution of CHO DNA stock (4-4×10⁶ RVLP copies) was made and the target segment was amplified with TaqMan Fast Virus 1-Step Mastermix containing the primer-probe set of interest. Reverse transcription and PCR amplification was carried out using the following thermal cycling conditions: Stage 1: 50° C. for 10 min, 1 cycle; Stage 2: 95° C. for 1 minute, 1 cycle; Stage 3: 40 cycles each consisting of: step 1: 95° C. for 3 seconds and step 2: 60° C. for 1 min.

Data generated from PCR amplification in Stage 1 and in Stage 2 were processed and analyzed to determine the relative performance of the various primer-probe sets.

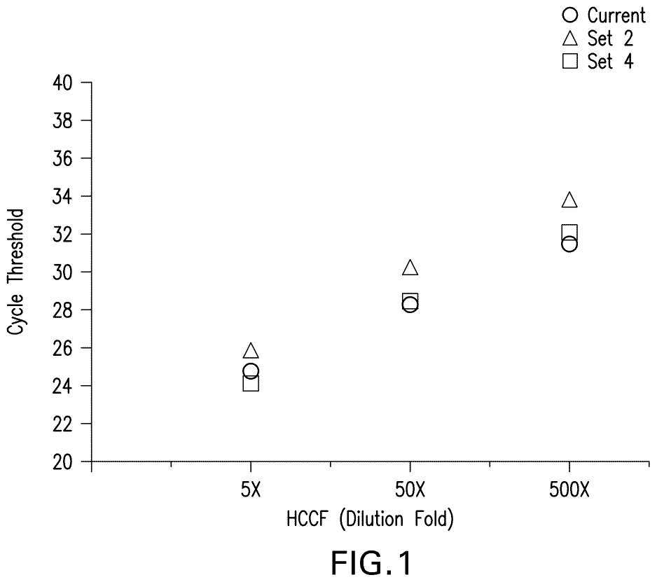

Performance of primer-probe sets 2, 4, and Current, with multiple dilutions of HCCF used as the sample, is shown in FIG. 1. The values for the cycle thresholds for the various samples are shown in Table 2 below.

TABLE 2

| | Cycle threshold | | |
|---|---|---|---|
| | HCCF dilution | | |
| Primer-Probe Set | 5X | 50X | 500X |
| Current | 24.76 | 28.27 | 31.47 |
| Set 4 | 24.42 | 28.62 | 31.71 |
| Set 2 | 25.86 | 30.25 | 33.81 |

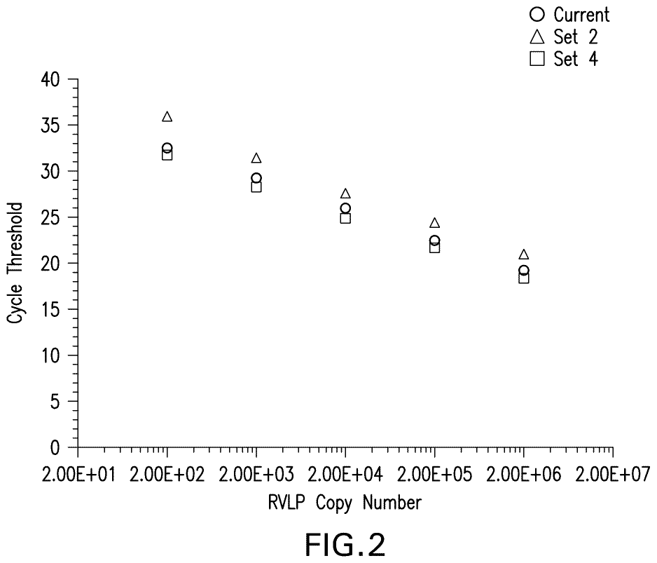

Performance of the same primer-probe, with different levels of CHO genomic DNA (as indicated by RVLP copy number) used as the sample, is shown in FIG. 2. The values for the cycle thresholds for the various samples are shown in Table 3 below.

TABLE 3

| | Cycle Threshold | | |
|---|---|---|---|
| RVLP Copy number | Current | Set 2 | Set 4 |
| 2.00E+06 | 19.23 | 20.98 | 18.35 |
| 2.00E+05 | 22.46 | 24.42 | 21.66 |
| 2.00E+04 | 25.97 | 27.57 | 24.87 |
| 2.00E+03 | 29.27 | 31.44 | 28.27 |
| 200 | 32.51 | 35.94 | 31.73 |

All primer-probe sets were observed to amplify their respective targets of interest. When compared to the primer-probe set, Current (DeWit et al.2000), primer-probe set 2 appears to be less sensitive while primer-probe set 4 appears to be equally or slightly more sensitive.

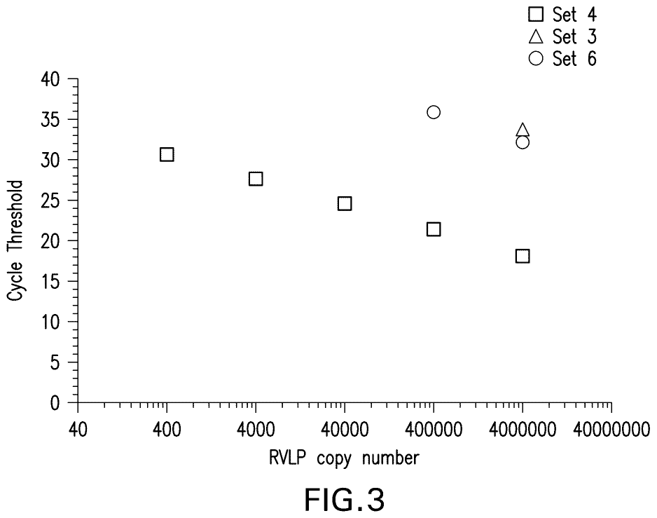

The remaining four primer-probe sets, i.e., sets 1, 3, 5, and 6, each targeting a different segment of RVLP genome within the pol region compared to primer-probe sets 4 and Current, were found to fail to amplify their targeted sequence (CHO genomic DNA being used as the sample) (see FIG. 3). Note that cycle threshold of 37 is taken to indicate lack of amplification, i.e., cycle thresholds above 37 are not shown in FIG. 3. Based on this result, primer-probe set 4 was selected for further experimentation and RVLP detection and quantitation.

Following the selection of primer-probe set 4, the RT-qPCR method was subjected to several rounds of optimization. The final method arrived at is a RT-qPCR that includes the steps: (1) extraction of RNA from RVLP particles in a sample, and (2) performing a one-step RT-qPCR to detect and quantitate the RNA copy number in the extracted sample. In step 1, 50 μL of the test sample was incubated with DNAse at 37° C. for 30 minutes to degrade CHO DNA. Next, the sample was digested with RNase and Proteinase K at 56° C. for 30 minutes to degrade RNA and protein, and the RNA from RVLP particles was chemically released using a high salt lysis buffer, followed by washing, and eluting using reagents supplied in PrepSEQ 1-2-3 Nucleic acid extraction kit (Applied Biosystem). In Step 2, 10 L of the eluant from Step 1 was combined with TaqMan Fast Virus 1-Step Mastermix, primer-probe set 4 (tagged with FAM), and ACTIN-probe (tagged with VIC), and the target of interest was amplified using the following thermal cycling conditions: Stage 1: 50° C. for 10 min, 1 cycle; Stage 2: 95° C. for 1 minute, 1 cycle; Stage 3: 40 cycles each consisting of: Step 1: 95° C. for 3 seconds; and Step 2: 55° C. to 60° C. for 60 seconds.

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

What is claimed is:

1. A method for detecting or quantifying retrovirus-like particles (RVLP) in a host cell culture using reverse transcription based real-time polymerase chain reaction (PCR) (RT qPCR), the method comprising (a) reverse transcribing genomic RNA from the RVLP to cDNA and (b) amplifying and quantifying the cDNA using a pair of primers and a probe, each primer pair and probe having sequences selected from the group consisting of:
(i)
(SEQ ID NO: 4)
5′-GGTAAGAGGCTCACCGTG-3′ (forward primer),
(SEQ ID NO: 5)
5′-GGTAAGCAGCCCCCTCCG-3′ (reverse primer),
(SEQ ID NO: 6)
5′-GTCGTTATGCCTTTGCCACTGCCCAT-3′ (probe);
and
(ii)
(SEQ ID NO: 10)
5′-CCGAAATGGCGGCTCCCC-3′ (forward primer),
(SEQ ID NO: 11)
5′-GGGCTGGTGATTCAAGGAG-3′ (reverse primer),
(SEQ ID NO: 12)
5′-CGGCCAGGGGTTGCTTTTAAATGGG-3′ (probe).

2. The method of claim 1, wherein the host cell culture comprises Chinese Hamster Ovary (CHO) cells.

3. The method of claim 2, wherein the CHO cells are deficient in dihydrofolate reductase.

4. The method of claim 1, wherein the primer pair and probe have the sequences shown in step b(i).

5. The method of claim 1, wherein the primer pair and probe have the sequences shown in step b(ii).

6. The method of claim 1, wherein the host cell culture is used for expressing a protein.

7. The method of claim 6, wherein the protein is pembrolizumab.
